(19) United States
(12) Patent Application Publication
(10) Pub. No.: US 2003/0191433 A1
(43) Pub. Date: Oct. 9, 2003

(54) Breast pump

(76) Inventor: John Gilbert Prentiss, Pamplin, VA (US)

Correspondence Address: John G Prentiss, 28 Keener Lane, Rabun Gap, GA, 30568, US

(21) Appl. No.: 10/186,067

(22) Filed: Jun. 29, 2002

Related U.S. Application Data
(63) Continuation-in-part of application No. 09/583,511, filed on May 31, 2000, now Pat. No. 6,440,100.

(51) Int. Cl. A61M 1/06

(52) U.S. Cl. 604/74; 604/75

(57) ABSTRACT

A concealed apparatus for hands free breast milk pumping and collection includes one or more low profile nipple caps held in place beneath a support brassiere or other minimally conspicuous confinement. The system permits substantially concealed, hands free breast pumping in a public environment without any remarkable change in the user's visible appearance. Milk is expressed from the breasts through novel, low profile nipple caps linked to a membranous collection reservoir which is optionally designed for milk storage and delivery to the infant user.

### FIELD OF THE INVENTION

The present invention relates to breastmilk pumping and collection systems and components thereof, more specifically to those systems which may be used hands-free in public with elements of the systems concealed beneath clothing.

### BACKGROUND

This is a continuation in part of U.S. patent application Ser. No. 09/583511 bearing the title, “Concealed Apparatus for Hands Free Breast Milk Pumping and Storage”. The object of the present application is to claim embodiments not specifically covered by the previous application and to disclose in further detail the novel breast pumping system components.

Natural breastfeeding is widely accepted as the best way to nurture an infant. However, breastfeeding is not always possible for working mothers and other women who are not able to be present for their babies at every feeding. Because breastmilk is best for babies and breastfeeding requires ongoing practice to sustain optimum lactation, expression of breastmilk with a pump has been a widely accepted practice for many years. Breast pumps range from simple hand operated models that pump one breast at a time to a wide selection of electric models now in use, most of which simultaneously pump both breasts. Currently available breast pumps require exposing the breasts to view, much more than is typical with breastfeeding. Consequently, mothers who pump usually need a private place in which to express milk. This often presents a serious impediment to breastfeeding and may be a deciding point in a mother choosing not to breastfeed.

The most common contemporary breast pumping systems use silicone nipple cups fitted at the top of tubular bottles. One or two bottles are held by the mother in front of her breasts while a hand pump or remote vacuum source expresses milk into the bottles. Examples of this design include Larsson, U.S. Pat. No. 5,954,690 Alternating Suction Breast Pump; Niederberger, U.S. Pat. No. 5,810,772 Mother's Milk Pump; Silver, U.S. Pat. No. 5,797,875, Breast Pump Assembly and Method; and Lockridge, U.S. Pat. No. 5,720,722, These systems all require that the mother hold fairly cumbersome containers in front of her breasts for approximately 15 minutes while pumping, usually necessitating the use of a private room for milk expression. Because many work environments are not set up for mothers to have a private place to pump, these conspicuous systems may be extremely inconvenient, discouraging women from ever starting breastfeeding, or may cause them to eventually discontinue breastfeeding as it may not seem to be worth the trouble of having to express milk in a ladies room. In addition, time away from the workstation for pumping may be a problem with unsympathetic employers and busy employees. These systems further are not advantageous for use in public.

Good quality breast pumping systems are important to maintaining lactation and long term breastfeeding for many mothers. Breast pumps currently in use make breast pumping a stressful experience for many, reducing milk production and limiting a mother's freedom. Being able to pump in public substantially unnoticed will enable a mother to relax more while pumping and to enjoy greater freedom, thereby enhancing milk supply. The need to hold and manipulate the nipple cups and collection bottles, combined with stressful pumping environments where a mother may feel rushed, are often responsible for decreased lactation. Consequently, a mother may be forced to resort to full time formula feeding long before she would normally do so.

Hands free pumping affords a mother the ability to simultaneously massage her breasts to enhance milk let down, a procedure that is not possible with most pumps currently in use. To mitigate the disadvantage of holding the collection containers while pumping, several inventions have been patented. These include Penny, U.S. Pat. No. 6,004,186, Apparatus for Securing Suction Devices to a Nursing Mother's Breasts; Lockridge, et.al. U.S. Pat. No. 5,575,768, Device and Kit for Supporting a Breast Shield and Related Pump Equipment; and Silver, et. al., U.S. Pat. No. 5,514,166, Device and Method for Supporting a Breast Shield and Related Pump Equipment. These systems are adaptations of the previously mentioned pumping units, and they leave the somewhat cumbersome collection bottles still suspended in front of the mother's breasts.

A departure from the above systems was patented by Jelks, U.S. Pat. No. 5,616,125. This design places the collection bottles along with pumping gear in a backpack linked to the breasts by tubes. While there are several reasons to question this system's functionality as disclosed, it is noteworthy. It is unclear how the vacuum lines pass through the T-shirt, or how those same lines can enter the apex of the domed nipple cups as specified and be held in place by a T-shirt. They certainly would not remain undetected in public, though no claim is made as to suitability for public use. The lines linking the breasts to the collection bottles are too long to clean easily and would likely need to be discarded after each use. Other drawbacks of Jelk's system include the backpack itself which seems to be cumbersome. Also, milk passes through the pumps before reaching the collection bottles, posing a sanitation and cleaning problem. The system remains unsuitable for anything but covert use, and does not address the principle objects of the present invention.

When 19^{th} century women wore corsets covering a majority of the torso, leakage from lactating breasts presented a major problem. To contend with this issue, Daniels, U.S. Pat. No. 166,686 discloses nipple cups to be worn beneath a corset or brassiere with drain lines extending to a remote reservoir. Milk naturally dripping from the breast was thereby prevented from soiling undergarments. More recently, Canadian Patent #2,033,604 was issued to Huynh for an under-bra collection system comprising a low profile nipple cap connected to a collection bottle located below each breast to catch leakage. However, Huynh's design makes no provision for breast pumping and is solely for replacing breast pads.

Breast shells were commonly used in the 19^{th} century to cope with leaking breasts and were later adapted to conceal public breast feeding in a Victorian society. Davidson, U.S. Pat. No 22,018, incorporates a breast shell with a tube having a nipple on the end so that an infant can draw milk from the breast while the mother is fully clothed. A similar milk expressing system was patented by Lewis, U.S. Pat. No. 22,080 a week later. The mother could use mouth suction to express small amounts of milk from her breast, then installs a nipple on the same tube and feed her baby from the breast shell. Although these systems may have made breast feeding in public more convenient, they did not provide for expressing and storing quantities milk for later use when the infant and mother could not be in the same location. Expanding on these two designs, Cunningham, U.S. Pat. No. 949,414 added a brassiere, but the system seems nearly impossible to conceal and ill suited for public use.

Also in the 19^{th} century, Cole, U.S. Pat. No. 72,604, invented a breast pump having a relatively low profile nipple cup connected to a squeeze bulb via rubber lines. This system was clearly not intended to be worn beneath clothing, as the stated method of operation instructs the user to hold the nipple cup in place while operating the pump “with the other hand”. There is no provision for holding the nipple cup in position other than manually, and it is suitable for pumping only one breast at a time. This design is extremely unsanitary since expressed milk had to pass through the squeeze bulb and a long span of latex tubing before reaching the collection vessel. Adequately cleaning these parts would be nearly impossible.

### SUMMARY OF THE INVENTION

The present system expands on the prior application to provide a hybrid system that is more efficient, comfortable, compact, convenient and versatile. In the present preferred embodiments, a rigid flask is not required for collection and storage because the prime mover drives the milk flow from a location upstream from the collection vessel. Overall air volume in the system is minimized to increase efficiency and reduce pump requirements. Additional new features includes the augmentation of pulsating pressure directly against the lactiferous glands of the breasts while negative pressure is applied to the nipples tip to assist the expression of milk. The present system allows milk to be conveyed to bag-type reservoirs from the breasts without contamination of the milk and without use of a rigid collection vessel. The present nipple caps have expanded function with enhanced performance, yet remain easy to clean and provide a minimally obtrusive profile when inserted beneath a brassiere or custom support system designed to mask and secure the nipple caps under the user's outer clothing. Any suitable pulsating vacuum or pressure source can be used to develop appropriate positive and negative pressure in the system to express milk from the breasts.

Significant variations on the present theme will be appropriate to provide comfortable, non-obtrusive system elements for a wide spectrum of body types. In addition, a built-in solenoid-type prime mover may be added to the nipple cap to apply pressure on the lactiferous ducts while a vacuum is simultaneously applied to the nipple. The present pumping system is unobtrusive to the extent that a mother can express breastmilk substantially unobserved while sitting at her desk at the office, traveling in a car, or even while using public transportation.

Objects and Advantages:

A principle object of the present invention is to provide a compact breast pumping apparatus which may be concealed beneath clothing and which facilitates the use of membranous collection, storage and dispensing bladders, thereby making breastfeeding a more convenient, more hygienic and less costly option for mothers who express breastmilk. A second object is to increase pumping efficiency by minimizing the work required by the prime mover. A third object is optionally to provide pulsating pressure directly to the lactiferous ducts to add comfort, to reduce the amount of suction required at the nipple, and to more closely imitate the action of a suckling infant. A fourth object is to obviate the need for a rigid collection vessel. A fifth object is to provide a breast pumping system in which non-stationary walls of a biased chamber in the nipple cap are actuated to exert positive pressure against the lactiferous glands while exerting a negative pressure on the nipples to express milk from the breasts. A sixth object is to provide a breastmilk collection, storage and delivery bag for the subject invention which may be used directly in certain infant feeding delivery system.

### BRIEF DESCRIPTION OF THE DRAWINGS

FIG. 1 is a frontal view of the first embodiment of the present invention;

FIG. 2 is a section of the low-profile nipple cap of FIG. 1

FIG. 3 is a frontal view of an alternate embodiment of the present invention;

FIG. 4 is a section of the vacuum interface chamber of FIG. 3;

FIG. 5 is a section of the low-profile nipple cap of FIG. 3

FIG. 6 details the collection bag of FIG. 1;

FIG. 7 is a section of an in-line solenoid pump.

### DETAILED DESCRIPTION OF THE DRAWINGS

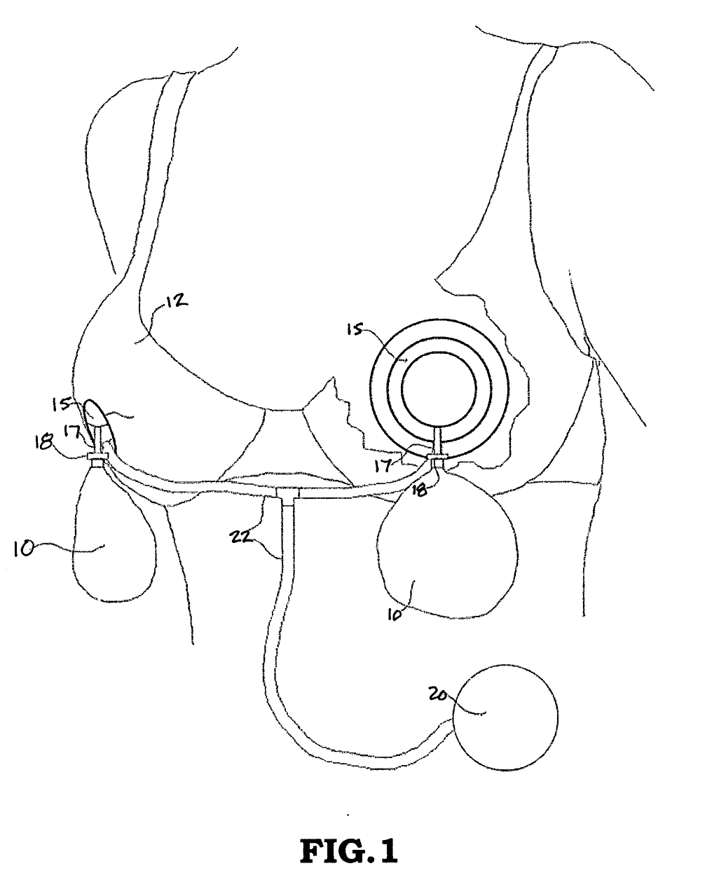

The FIG. 1 depicts the subject invention employing membranous collection bags 10 suspended beneath the breasts which are supported by a custom brassiere 12 shown cutaway over the left breast to reveal nipple cap 15. Collection bags 10 are suspended via milk conduits 17 which are securely, but removably attached to nipple caps 15. Line valves 18 are provided in each milk conduit 17 to prevent reflux of the milk. Milk conduits 17 pass through holes 19 in brassiere 12 to convey milk from the nipple caps 15 to collection bags 10, which may be suspended thereby as shown. The system is light weight and will typically be used sitting down, requiring nominal support for collection bags 10. Further support for collection bags 10 may optionally be provided by exterior pockets (not shown) in brassiere 12, by straps, or by other suitable means. Milk conduits 17 in this embodiment are of neoprene or other FDA approved material, approximately 1 mm or greater inside diameter. Nipple caps 15 are held firmly against breasts by brassiere 12 which is low cut for easy insertion and adjustment of nipple caps 15 without removal.

The external prime mover 20 shown in the figured embodiment is a simple elastomeric squeeze bulb but may schematically represent any other suitable prime mover, depending on the design requirements of nipple cap 15. When squeezed, prime mover 20 produces pressure and vacuum communicated through actuator conduits 22 which are tubular fluid conduits adequate to withstand positive or negative pressure generated by prime mover 20. Prime mover 20 may be an electric pump, manual pump or other means capable of developing a pulsating force to initiate the milk expression action of nipple cap 15, the related criteria being well know to those versed in the art.

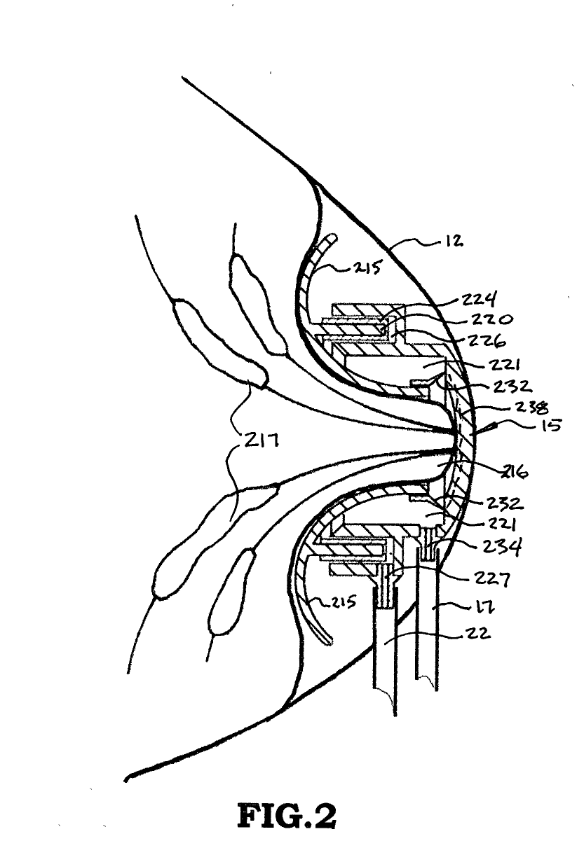

FIG. 2 is a section of the low profile nipple cap shown in FIG. 1. Nipple cap assembly 15 is held securely against the breast by brassiere 12. Conical flange 215 is sized and shaped to receive nipple 216 while compressing the breast in the area above lactiferous glands 217. Conical flange 215 is integral with annular piston member 220 which is capped with annular seal 224. Piston member 220 and annular seal 224 are sized to sealingly slide in and out of piston cavity 226. When pressure is applied to annular piston cavity 226 via port 227 and conduit 22, piston 220 is forced against lactiferous glands 217 while vacuum cavity 221 is elongated, thereby increasing the volume of vacuum cavity 221 and developing a negative pressure therein to express milk from nipple 216. Milk is drawn past valvular flange 232 through vacuum chamber 221 and out via milk port 234 and milk conduit 17. While valvular flange 232 provides some protection against reflux, a second reflux line valve 18 is recommended between milk port 234 and the milk collection bag 10 of FIG. 1. Detents 238 are provided at the inner extremity of vacuum cavity 221 to prevent blockage of milk ducts the tip of nipple 216. Biasing means such as a coil spring (not shown) may be installed in annular piston cavity 226 to cause piston 220 to be normally distended so that optionally vacuum may then be used instead of pressure to pull piston 220 and conical flange 215 into the position shown in the figured embodiment. When the vacuum is released, piston 220 is driven by the spring against lactiferous glands 217 and negative pressure is again created in vacuum chamber 221 by volumetric expansion.

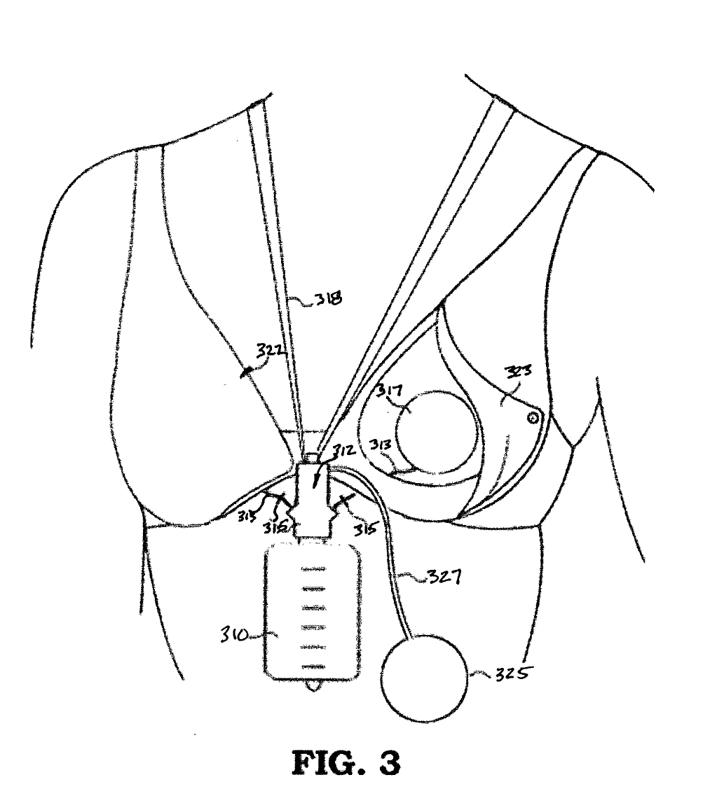

In FIG. 3, collection bag 310 is suspended beneath vacuum interface assembly 312. Milk lines 313 each have in-line reflux valves 314 and carry milk between nipple cap 315 and vacuum interface assembly 312. The vacuum interface assembly 312 and collection bag 310 are jointly suspended from neck strap 318. As is the FIG. 1 embodiment, nipple cap 317 is held firmly against the breast by brassiere 322 when flap 323 is in the closed position as shown on the right breast. Prime mover 325 develops vacuum in this instance which is communicated through tubular conduit 327 to vacuum interface assembly 312.

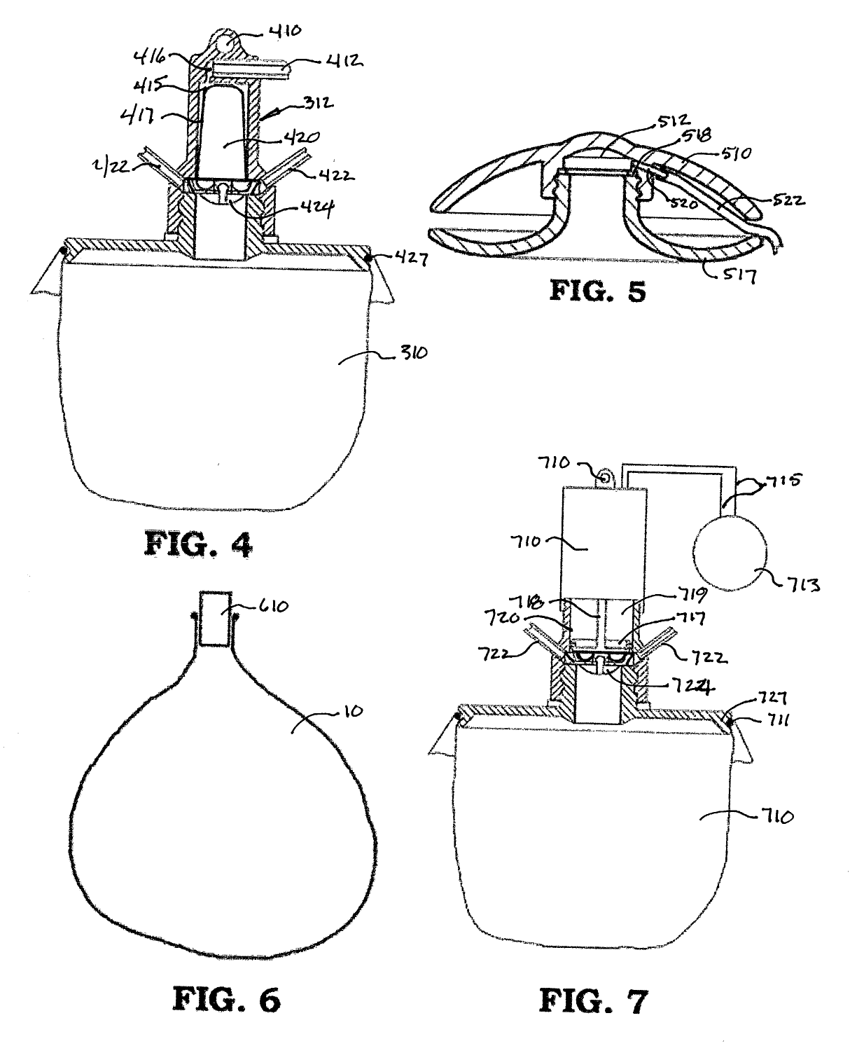

FIG. 4 is a section of vacuum interface assembly 312 of FIG. 3. The assembly 312 is suspended from the user's neck by strap 318 of FIG. 3 which hooks into hole 410 at the top of the assembly 312. Vacuum conduit 412 communicates with first vacuum chamber 415 via port 416 to pull resilient diaphragm 417 upward as shown into the fully distended position. Vacuum is communicated via diaphragm 417 to second vacuum chamber 420. Vacuum is thence communicated to the nipple caps (see FIG. 5) via milk conduits 422. Valve 424 is situated downstream from milk conduits 422, interfacing between second vacuum chamber 420 and milk collection bag 310 which is secured by elastic band around bag flange 427. In operation, valve 424 closes with negative pressure in second vacuum chamber 420, and opens with positive pressure to permit milk to enter collection bag 310 Alternatively, a piston (not shown) may be substituted for diaphragm 417 and may be spring biased to permit the system to use a pressure rather than vacuum source to actuate the system. A non-permeable, moveable interface such as diaphragm 417 serves to fully isolate the prime mover from the milk flow, minimizing contamination in the milk and likewise in the prime mover. However, since gravity will serve to separate the milk from the vacuum system in this embodiment in most instances, diaphragm 417 is non-essential to the operation of the system.

FIG. 5 details nipple cap 315. Annular outer shell 510 includes cavity 512 and threaded receptacle 515 which receives threaded flange 517. Gasket 518 is provided to seal cavity 512 when flange 517 is installed over the fore portion of a human female breast. Port 520 communicates with milk conduit 522. In operation, vacuum is communicated via milk conduit 422 to draw a vacuum on the nipple in cavity 512. By pulsating and releasing the vacuum, flange 517 is repeatedly drawn against the breast to stimulate the lactiferous glands while vacuum in cavity 512 further serves to express milk from the breast. Milk is carried by vacuum and gravity to the collection vessel via milk conduit 422.

FIG. 6 details a membranous collection, storage and dispensing bag 10 used in the FIG. 1 embodiment but fully adaptable to other embodiments of the subject invention. In this instance, bag 10 may be fabricated of dipped latex or any FDA compliant membranous thermoplastic, thermoplastic elastomer or thermoset elastomer. Particularly where flimsy materials are used, tube 610 may be sealingly inserted into the neck of bag 10 to lend rigidity to the opening. Tube 610 may be slightly tapered on both ends and may be sized approximately 12 mm outside diameter at mid-point to sealingly be inserted into a resilient infant feeding nipple for direct dispensing to an infant. The neck of bag 10 is sized accordingly at approximately 11 mm inside diameter. Bag 10 may be disposable or reusable and may be closed with a valve (not shown) within tube 610 or otherwise.

FIG. 7 depicts an example of use of a solenoid actuator 710 as a concealed element in the system. As in FIG. 4, hole 710 is available to support the assembly from the user's neck. Solenoid 710 is actuated by remote power source 713 shown schematically, via electrical wires 715. Piston 717 is affixed to armature 718 and is driven by solenoid 710 to sealingly slide in cylinder 720, creating a vacuum in chamber 722 on the upstroke. Vacuum is thence communicated to the nipple caps (see FIG. 5) via milk conduits 722. Valve 724 is situate downstream from milk conduits 722, interfacing with milk collection bag 710 which is secured by elastic band around bag flange 727. In operation, valve 724 closes when negative pressure in chamber 422 is created by actuation of solenoid 710 which pulls piston 717 upward in cylinder 720. Valve 724 opens with positive pressure on the down stroke of piston 717 to permit milk to enter collection bag 710. While in this embodiment upper cavity 719 is vented, pressure created on the upstroke could be readily communicated to the FIG. 2 nipple cap embodiment. Applying a pulsating or continuous vacuum to the system imitates a suckling infant, thus expressing milk from the breasts to the reservoir for later use.

Ramifications and Scope:

Numerous alternatives to the figured embodiments exist for the construction of the subject breastmilk pumping and collection system. The prime mover may be any suitable manual or electric pump or mouth suction produce either a vacuum or pressure. The pressure or vacuum interface on the system may be any means to interface the milk expression and vacuum systems such as a piston, diaphragm, membrane or simply gravitational separation. Vacuum as well as pressure may be applied to the interface from any appropriate source. To use pressure instead of vacuum, the piston or diaphragm interface is biased so that pressure loads the system and when released, vacuum is created on the milk expression side by the biasing of the membrane or piston. Biasing may include compression of the breast tissue, a mechanical spring, an elastic membrane or other suitable means. Materials and method of manufacture may also vary widely while remaining within the spirit of the invention, as components may be both disposable or non-disposable, flexible and rigid, one piece or multiple pieces, or units having external or internal ribs. Custom liner bags may include an integral nipple or nipple port for direct infant feeding. Design alternatives for brassieres or other means to support the elements of the system are extensive. System elements such as the pump and collection bag may be located beneath the breasts, between the breasts, at the waist, at the nipples, remote from the body or elsewhere convenient to the user.

Those skilled in the art will appreciate that the concepts disclosed herein may readily inspire other concealed embodiments fulfilling the objectives of the present invention. It is therefore intended that the claims be regarded as including such equivalent constructions insofar as they do not depart from the spirit and scope of the present invention. The appended drawings depict preferred embodiments without intending to limit the scope of the invention which is more fully delineated by the claims that follow.

I claim:

1. A breast milk pumping and collection system for expressing and collecting human milk, the improvements comprising concealed components wherein at least one nipple cap covers the nipple portion of a human female breast presenting a low external profile to permit inconspicuous use beneath outer garments, said breast milk pumping and collection system including support means to press said nipple cap sealingly against said breast so as to be substantially concealed beneath said outer garments, said nipple cap comprising a hollow cavity having outside and inside surfaces to surround and enclose a nipple drawn out by vacuum without blocking flow from milk ducts at the tip of said nipple, said hollow cavity further including: a.) an annular opening through which said nipple is received, b.) a substantially conical flange portion coaxial with said annular opening, said flange portion serving to cushion said breast while stimulating the lactiferous glands beneath the areola of said breast and providing a vacuum seal between said breast and said annular opening; c.) a milk delivery port communicating with said hollow cavity, said milk delivery port further communicating with a milk collection reservoir; d.) means to develop a negative pressure in said hollow cavity and e.) said milk collection reservoir.

2. An annular nipple cap to cover the fore portion of a human female breast, said nipple cap being of low profile and comprising a hollow cavity having outside and inside surfaces to surround and enclose a nipple providing room in said hollow cavity to permit said nipple to be distended by vacuum without blocking flow from milk ducts at the tip of said nipple, said hollow cavity further including a) an annular opening through which said nipple is received, b) a substantially conical flange portion, said flange portion serving to cushion said breast while stimulating the lactiferous glands beneath the areola of said breast and providing a vacuum seal between said breast and said annular opening, and c. a milk delivery port communicating between said hollow cavity and a breastmilk collection reservoir.

3. The breast milk pumping and collection system of claim 1 wherein said collection reservoir is a bladder-type collection bag.

4. The breast milk pumping and collection system of claim 1 wherein said collection bag is disposable.

5. The breast milk pumping and collection system of claim 1 wherein said collection bag can be used as a delivery system for infant feeding.

6. The breast milk pumping and collection system of claim 1 wherein said means to develop a negative pressure in said hollow cavity first interfaces with the milk flow path at a central location between the breasts.

7. The breast milk pumping and collection system of claim 1 wherein said means to develop a negative pressure in said hollow cavity first interfaces with the milk flow path at said nipple cap.

8. The breast milk pumping and collection system of claim 1 wherein said means to develop a negative pressure in said hollow cavity is selected from a list of prime movers including an electric pump, a hand operated pump, an arm operated pump, a leg operated pump, a foot operated pump, a squeeze bulb pump, a concealed pump, a non-concealed pump, a remote pump, a pulsing solenoid pump, a diaphragm pump, a piston pump, a rotary pump, an alternating current electric pump, a direct current electric pump, and mouth suction.

9. The breast milk pumping and collection system of claim 1 wherein said means to develop a negative pressure in said hollow cavity is selected from a list including vacuum from a remote source, vacuum from a local source, pressure applied to non-stationary walls of a biased chamber which in turn communicates a negative pressure to said hollow cavity.

10. The breast milk pumping and collection system of claim 1 wherein means are included to exert positive pressure against the lactiferous glands while exerting a negative pressure on the nipples to express milk from the breasts.

11. The breast milk pumping and collection system of claim 1 wherein a negative pressure is created in said hollow cavity via said milk delivery conduit.

12. The breast milk pumping and collection system of claim 1 wherein a negative pressure is created in said hollow cavity via means other than said milk delivery conduits.

13. A membranous milk collection bag having a more rigid fill port approximately 12 mm in outside diameter, said fill port being sized to sealingly fit inside the resilient nursing nipple of a breast-type feeding container for direct dispensation to a user infant, said membranous milk collection bag containing a volume of approximately 50 ml to 200 ml consistent with typical breastmilk production.

14. The breast milk pumping and collection system of claim 1 wherein said collection reservoir is located in a position including beneath the breasts, between the breasts, at the waist, under the arms, in pockets in a brassiere and in a remote location.

15. The breast milk pumping and collection system of claim 1 wherein said means to press said nipple cap sealingly against said breast is selected from a list including various embodiments of a brassiere, a halter, a corset, a girdle, an elastic band encircling the chest, and an outer garment.

16. The breast milk pumping and collection system of claim 1 wherein said collection reservoir is located outside the user's clothing and is not concealed.

17. The nipple cap of claim 2 wherein means are included to exert positive pressure against the lactiferous glands while exerting a negative pressure on the nipples to express milk from the breasts.

18. The nipple cap of claim 2 wherein said collection reservoir is placed in a location selected from a list including: beneath the breast, between the breasts, integral with said support means, at the user's waist, and in a location apart from the user.

19. The nipple cap of claim 2 wherein said means to develop a negative pressure in said hollow cavity is selected from a list including vacuum from a remote source, vacuum from a local source, pressure applied to non-stationary walls of a biased chamber which in turn communicates a negative pressure to said hollow cavity.

20. The nipple cap of claim 2 wherein said means to develop a negative pressure in said hollow cavity is selected from a list of prime movers including an electric pump, a hand operated pump, an arm operated pump, a leg operated pump, a foot operated pump, a squeeze bulb pump, a concealed pump, a non-concealed pump, a remote pump, a pulsing solenoid pump, a diaphragm pump, a piston pump, an alternating current electric pump, a direct current electric pump, and mouth suction.
